(19) United States
(12) Patent Application Publication
(10) Pub. No.: US 2025/0327096 A1
(43) Pub. Date: Oct. 23, 2025

(54) METHOD FOR ENHANCING GROWTH OF DUNALIELLA SPECIES AND BIOMASS PRODUCTION THEREFROM

(71) Applicant: THE HONG KONG POLYTECHNIC UNIVERSITY, Hong Kong (CN)

(72) Inventors: Hang-kin KONG, Hong Kong (CN); Chun-Lap Samuel LO, Hong Kong (CN); Sirius TSE, Hong Kong (CN); Wing-Tak WONG, Hong Kong (CN); Ka Fu YUNG, Hong Kong (CN)

(21) Appl. No.: 18/642,902

(22) Filed: Apr. 23, 2024

(51) Int. Cl. C12P 1/00; C12N 1/12; C12N 1/20; C12P 39/00; C12R 1/01; C12R 1/89

(52) U.S. Cl. CPC C12P 1/00; C12N 1/125; C12N 1/205; C12P 39/00; C12R 2001/01; C12R 2001/89

(57) ABSTRACT

A method for enhancing growth of a microalgal species and its biomass production yield by co-cultivating the microalgal species with a bacterial isolate under the genus of Pseudoalteromonas in separate compartments. The microalgal species is selected from Dunaliella species. The genome sequence of the Pseudoalteromonas isolate can have a nucleotide similarity of lower than 95% to that of other Pseudoalteromonas species.

### REFERENCE TO SEQUENCE DISCLOSURE

A sequence listing file with a file name “P25477US00_sequence_listing.xml” in ST.26 XML file format having a file size of 4,455 KB created on Sep. 13, 2023 is incorporated herein by reference in its entirety.

### REFERENCE TO A DEPOSIT OF BIOLOGICAL MATERIAL

This application contains a reference to a deposit of biological material, which deposit is incorporated herein by reference.

### TECHNICAL FIELD

The present disclosure relates to a method for enhancing the growth rate and yield of biomass from Dunaliella spp. In particular, the method includes co-cultivating the Dunaliella spp. with a novel Pseudoalteromonas isolate.

### BACKGROUND

Microalgae are well-known species that can produce useful biomass and extracts. Among which, marine microalga, Dunaliella spp., has been used commercially to produce feedstock for farmed animals, biofuel production, and also as a dietary supplement (e.g., beta-carotene) as it is one of the few species recognized by the FDA as Generally Recognized as Safe (GRAS). It is also believed that microalgal cultivation can lower CO₂ emission. Therefore, methods for enhancing the growth and yield of Dunaliella spp. and biomass production has drawn a lot of attention.

However, the traditional way to boost the growth rate of microalgae usually involves using high concentrations of substrate in the culture medium which costs more than regular growth. Separation of useful materials from other materials in a more condense microalgal culture is another issue as the traditional separation process is normally labor-intensive or low yield of biomass which might not justify the high operational cost.

Some studies suggested optimizing the culture conditions, such as CO₂ content, light intensity and wavelength for Dunaliella spp. to increase the biomass yield and change the composition of the by-products (Jeon et al., “Enhanced production of biomass and lipids by supplying CO₂ in marine microalga Dunaliella sp.” J Microbiol. 51, 773-776 (2013); Sui et al., “Effect of Light Intensity and Wavelength on Biomass Growth and Protein and Amino Acid Composition of Dunaliella salina.” Foods 2021, 10(5), 1018).

Some other studies proposed that co-culture of microalgae with other genus of microbes such as yeast or bacteria, or even with a different microalgal species may enhance the growth and yield of biomass from the microalgal culture. For instance, a mutualistic microalga-bacteria consortium is based on the exchange of resources and services between two different types of microbes, i.e., the bacteria utilize the organic carbon and dissolved oxygen released from algal photosynthesis, in return, supply nutrients, vitamins, trace elements and carbon dioxide for microalgal growth. They co-exist and have symbiotic interactions with each other. It has been found that some microalgae show higher interaction with bacteria than the others. For instance, Samo et al. (Attachment between heterotrophic bacteria and microalgae influences symbiotic microscale interactions. Environ. Microbiol. 2018, 20, 4385-4400) reported that Phaeodactylum tricornutum had attached bacteria and fixed 64% more carbon than axenic cells, whereas Nannochloropsis salina had only 42-63% of the cells associated with bacteria and just fixed 10% more carbon compared to axenic cells. They also showed that an uncultured bacterium related to Haliscomenobacter sp. increased carbon fixation in P. tricornutum and incorporated 71% more fixed C than other bacteria.

In addition, the co-cultivation parameters, such as number of individual populations, degree of separation among different populations, volume of cultures, timeline of the co-cultures, etc. would directly impact the outcome of the interaction. Approaches of co-cultivation would also affect the efficiency of the co-cultivation. For instance, one of the most commonly used setups in co-cultivation studies is suspension. Other common experimental designs include immobilizing one type of microbes as biofilms while suspending the others in a vessel, using different carriers to encapsulate different microbes, or separation of two different microbes by a membrane. Through careful selection of two different species or strains not interacting or co-existing in nature and optimizing the co-cultivation conditions for the selected species/strains, a synthetic mutualism is achievable in order to enhance the growth of the target species/strain in a well-controlled and sustainable manner. Besides co-cultivation, extracts from one of the microbes can be a source of growth factors to enhance the growth of other microbes.

There thus exists a need to developed improved methods for cultivating Dunaliella spp. that address or overcome at least of the shortcomings addressed above.

### SUMMARY

Accordingly, an aspect of the present disclosure proposes a method for co-culturing a microalgal species under a genus of Dunaliella with a bacterial strain under a genus of Pseudoalteromonas in a confined co-cultivation environment to enhance growth of the microalgal species comprising providing the microalgal species and the bacterial strain to the confined co-cultivation environment, providing a culture medium to the confined co-cultivation environment and a set of co-cultivation conditions.

In certain embodiments, the microalgal species comprises Dunaliella tertiolecta (CCMP364 isolated from Norway or CCMP1320 with unknown origin) or a species named STK2. STK2 is a Dunaliella sp. isolated from Sha Tau Kok, Hong Kong in 2013.

In certain embodiments, the bacterial strain comprises an isolate under the genus of Pseudoalteromonas having a genome sequence composed of two circular DNAs represented by SEQ ID NOs: 1 and 2, respectively.

In certain embodiments, the bacterial strain further comprises any species under the genus of Pseudoalteromonas having a genome sequence with a nucleotide similarity of at least 90% to SEQ ID NOs: 1 and 2.

In certain embodiments, the species having the genome sequence with the nucleotide similarity of at least 90% to the genome sequence of the isolate comprises Pseudoalteromonas shioyasakiensis, Pseudoalteromonas gelatinilytica, or Pseudoalteromonas piscicda.

In certain embodiments, the bacterial strain comprises the Pseudoalteromonas having been deposited with the American Type Culture Collection under Patent Deposit Designation Number PTA-127701.

In certain embodiments, the confined co-cultivation environment comprises at least two compartments each accommodating the microalgal species or the bacterial strain, and the compartment accommodating the bacterial strain is configured to be permeable to substances of less than 10 kDa in molecular weight between the two compartments accommodating the microalgal species and the bacterial strain, respectively.

In certain embodiments, the culture medium comprises silicate-free K medium or L1 medium.

In certain embodiments, the set of co-cultivation conditions comprises culture temperature is 22-23° C. Light intensity is 7-8 μmole m−2 s−1, under a 12:12 h light:dark cycle. Partial pressure of CO₂ is 400 ppm.

In certain embodiments, the microalgal species and the bacterial strain are provided in a cell density of 1:1000.

A further aspect of the present disclosure provides an algal growth enhancing composition comprising a biological agent or an extract thereof, where the biological agent comprises a genome sequence composed of two circular DNAs represented by SEQ ID NOs: 1 and 2, respectively, or any genomic sequence having a nucleotide similarity of at least 90% to the genome sequence represented by SEQ ID NOs.: 1 and 2.

In certain embodiments, the biological agent is a bacterial strain under a genus of Pseudoalteromonas.

In certain embodiments, the bacterial strain comprises Pseudoalteromonas shioyasakiensis, Pseudoalteromonas gelatinilytica, Pseudoalteromonas piscicda, or any Pseudoalteromonas spp. comprising a genomic sequence having a nucleotide similarity of at least 90% to the genome sequence represented by SEQ ID NOs: 1 and 2.

In certain embodiments, the bacterial strain comprises the Pseudoalteromonas having been deposited with the American Type Culture Collection under Patent Deposit Designation Number PTA-127701.

In certain embodiments, the algal growth is a growth of microalgal species under a genus of Dunaliella.

In certain embodiments, the microalgal species comprises Dunaliella tertiolecta (CCMP364 isolated from Norway or CCMP1320 with unknown origin) or a species named STK2. STK2 is a Dunaliella sp. isolated from Sha Tau Kok, Hong Kong in 2013.

In certain embodiments, the extract of the biological agent comprises a culture medium, cellular and molecular contents of the biological agent.

In certain embodiments, the extract has a molecular weight of less than 10 kDa.

A further aspect of the present disclosure provides a method for increasing biomass production yield from a microalgal species comprising co-culturing the microalgal species with a bacterial strain under a genus of Pseudoalteromonas, wherein the bacterial strain comprises a genome sequence composed of two circular DNAs represented by SEQ ID NOs: 1 and 2, respectively, a genome sequence having a nucleotide similarity of at least 90% to the genome sequence represented by SEQ ID NOs: 1 and 2, or the bacterial strain comprises the Pseudoalteromonas having been deposited with the American Type Culture Collection under Patent Deposit Designation Number PTA-127701. Alternatively, the method for increasing the biomass production yield from the microalgal species comprising contacting the microalgal species with an extract of the bacterial strain described herein, where the extract comprises a culture medium, cellular and molecular contents of the bacterial strain.

In certain embodiments, the microalgal species comprises Dunaliella tertiolecta (CCMP364 isolated from Norway or CCMP1320 with unknown origin) or a species named STK2. STK2 is a Dunaliella sp. isolated from Sha Tau Kok, Hong Kong in 2013.

In certain embodiments, the bacterial strain comprises Pseudoalteromonas shioyasakiensis, Pseudoalteromonas gelatinilytica, Pseudoalteromonas piscicda, or any Pseudoalteromonas spp. comprising a genomic sequence having a nucleotide similarity of at least 90% to the genome sequence represented by SEQ ID NOs: 1 and 2.

In certain embodiments, the bacterial strain comprises the Pseudoalteromonas having been deposited with the American Type Culture Collection under Patent Deposit Designation Number PTA-127701

In certain embodiments, said co-culturing is performed in a confined co-cultivation environment.

In certain embodiments, the confined co-cultivation environment comprises at least two compartments each accommodating the microalgal species or the bacterial strain, and the one accommodating the bacterial strain is configured to be permeable to substances of less than 10 kDa in molecular weight between the two compartments accommodating the microalgal species and the bacterial strain, respectively.

In certain embodiments, the extract has a molecular weight of less than 10 kDa.

A further aspect of the present disclosure provides a co-culture system comprising a microalgal species under the genus of Dunaliella and an isolate of bacterial strain under the genus of Pseudoalteromonas, where the isolate of bacterial strain under the genus of Pseudoalteromonas comprises a genome sequence composed of two circular DNAs represented by SEQ ID NOs: 1 and 2 or the bacterial strain comprises the Pseudoalteromonas having been deposited with the American Type Culture Collection under Patent Deposit Designation Number PTA-127701.

This summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. Other aspects of the present disclosure are disclosed as illustrated by the embodiments hereinafter.

### BRIEF DESCRIPTION OF DRAWINGS

The appended drawings, where like reference numerals refer to identical or functionally similar elements, contain figures of certain embodiments to further illustrate and clarify the above and other aspects, advantages and features of the present invention. It will be appreciated that these drawings depict embodiments of the invention and are not intended to limit its scope. The invention will be described and explained with additional specificity and detail through the use of the accompanying drawings in which:

FIG. 1 shows the result of phylogenetic analysis between the Pseudoalteromonas isolate (ATCC Patent Deposit No. PTA-127701) of the present disclosure and other 37 species under the genus of Pseudoalteromonas based on their genomic DNA sequences for 12 Pseudoalteromonas spp. and long scaffolds (or assembles of long DNA sequences) for the remaining 25 species, where the arrow indicates where the present Pseudoalteromonas isolate (psl_c) is located in the phylogenetic tree, and the solid line box indicates the two closest relatives, P. shioyasakiensis and P. gelatinilytica;

FIG. 2 shows an average nucleotide similarity of the genomic data of 14 closer Pseudoalteromonas spp. to the present Pseudoalteromonas isolate (ATCC Patent Deposit No. PTA-127701) among the 37 species analyzed as shown in FIG. 1, where the percentage of similarities shown are based on BLASTn alignment; the solid line box indicates that none of the genome of 14 Pseudoalteromonas would have nucleotide similarity of >95% (indication of the same species) to the present Pseudoalteromonas isolate;

FIG. 3A shows the dose-response effect of living Pseudoalteromonas isolate (ATCC Patent Deposit No. PTA-127701) on microalgal density (cell count per ml) of D. tertiolecta CCMP364 after co-culturing for 14 days, where 1 unit=microalgae-bacteria ratio 1:1000;

FIG. 3B shows a dose-response curve from 0.1 to 1 unit of the living present Pseudoalteromonas isolate (ATCC Patent Deposit No. PTA-127701) on the microalgal densities of CCMP364 after the co-culturing for 14 days as in FIG. 3A;

FIG. 4 shows a growth performance curve of D. tertiolecta CCMP364 co-cultivated with the present Pseudoalteromonas isolate (ATCC Patent Deposit No. PTA-127701, PS1) according to certain embodiments compared to that without co-cultivation (Control);

FIG. 5 shows growth performance curves of D. tertiolecta CCMP364 under the cultivation with 5 times-diluted medium (0.2×medium) and 5-times-diluted medium with the present Pseudoalteromonas isolate (ATCC Patent Deposit No. PTA-127701)(0.2×medium+PS1) compared to that without medium-dilution or co-cultivation (C);

FIG. 6A shows a growth performance curve of D. tertiolecta CCMP364 co-cultivated with the living present Pseudoalteromonas isolate (ATCC Patent Deposit No. PTA-127701) in a 90-L inoculum level (Live PS1) compared to that without co-cultivation in the same inoculum level (Control);

FIG. 6B shows a comparison in dried weight of the biomass obtained from the co-culture of D. tertiolecta CCMP364 and the present Pseudoalteromonas isolate (ATCC Patent Deposit No. PTA-127701) grown in the 90-L medium and the biomass obtained from the control culture of D. tertiolecta CCMP364 alone grown in the 90-L medium as in FIG. 6A;

FIG. 7 shows growth performance curves of D. tertiolecta CCMP364 co-cultivated with P. piscicda (ATCC-15057) and P. distincta (ATCC-700518), respectively, and compared to that without co-cultivation (Control); and

FIG. 8 shows a workflow of a method for enhancing the growth of Dunaliella spp. and yield of biomass production therefrom according to certain embodiments of the present invention.

Skilled artisans will appreciate that elements in the figures are illustrated for simplicity and clarity and have not necessarily been depicted to scale.

### DETAILED DESCRIPTION

It will be apparent to those skilled in the art that modifications, including additions and/or substitutions, may be made without departing from the scope and spirit of the invention. Specific details may be omitted so as not to obscure the invention; however, the disclosure is written to enable one skilled in the art to practice the teachings herein without undue experimentation.

The present disclosure proposes a cultivation method to increase growth rates and yields (dried biomass) of Dunaliella spp. by co-culturing with a novel Pseudoalteromonas isolate in a confined cultivation environment with at least two compartments separated by a permeable membrane, e.g., the Pseudoalteromonas isolate is accommodated in a semi-permeable vessel configured to be permeable to substances of certain molecular weight, such that the Dunaliella spp. and the isolate do not have physical contact with each other. The present method is more cost-efficient for culturing Dunaliella spp. and obtaining a higher yield of biomass since the costs for culture medium and separation the present Pseudoalteromonas isolate from Dunaliella biomass before harvesting have been significantly reduced.

In certain embodiments, Dunaliella tertiolecta (CCMP364 isolated from Norway or CCMP1320 with unknown origin), which is purchased from the National Collection of Marine Algae and microbiota (NCMA), is provided. In certain embodiments, a microalgal strain named STK2 (Dunaliella sp.), which was isolated from Sha Tau Kok, Hong Kong in 2013, is provided. It belongs to the genus of Dunaliella, but the species of this isolate has never been identified.

In certain embodiments, the Pseudoalteromonas isolate which was isolated from a blooming event in Tai Mei Tuk, Taipo, Hong Kong in August 2012 by repeated marine agar plate spreading is provided.

In certain embodiments, the Pseudoalteromonas isolate has a genome composed of two circular DNAs, where one of them has 3,646,986 bp while the other has 912,021 bp which are represented by SEQ ID NOs: 1 and 2, respectively.

### Deposit of Biological Material

The following biological material has been deposited under the terms of the Budapest Treaty with the American Type Culture Collection (ATCC®) Patent Depository, 10801 University Boulevard, Manassas, Virginia 20110 USA, and given the following accession number:

| | ATCC Patent | |
|---|---|---|
| Deposit | Deposit Number | Date of Deposit |
| Pseudoalteromonas sp. | PTA-127701 | Mar. 7, 2024 |

The strain has been deposited under conditions that assure that access to the culture will be available during the pendency of this patent application and any patent issuing therefrom to one determined by foreign patent laws to be entitled thereto. The deposit represents a substantially pure culture of the deposited strain. The deposit is available as required by foreign patent laws in countries wherein counterparts of the subject application, or its progeny are filed. However, it should be understood that the availability of a deposit does not constitute a license to practice the subject invention in derogation of patent rights granted by governmental action.

In certain embodiments, the bacterial strain under the genus of Pseudoalteromonas comprises the Pseudoalteromonas having been deposited with the ATCC under Patent Deposit Designation Number PTA-127701.

The following examples accompanied with corresponding drawings are intended to better illustrate various embodiments of the present invention. Scope of the present invention should be defined in the appended claims.

### EXAMPLES

### Example 1—Phylogentic Analysis and Genomic Sequence Comparison of Pseudoalteromonas Isolate (ATCC Patent Deposit No. PTA-127701) with Other Pseudoalteromonas spp.

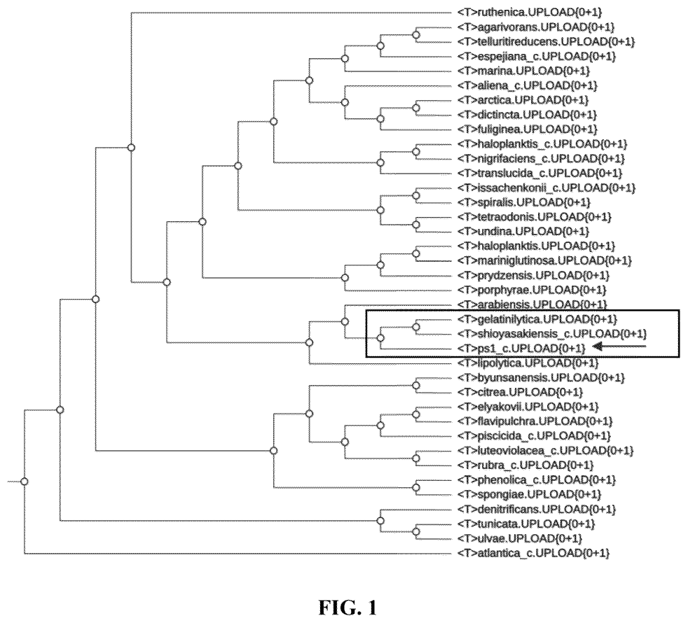

The two circular DNA sequences (SEQ ID NOs: 1 and 2) were used to identify the taxonomy of the isolate by phylogenetic analysis. Genomic DNA sequences or assembles of long DNA sequences of 37 Pseudoalteromonas spp. obtained from NCBI genome database and the two circular DNAs of the present isolate are clustered by Cytree 3. The results are shown in FIG. 1. As seen from FIG. 1, it was suggested that the isolate belongs to the genus of Pseudoalteromonas. The two closest relatives to the isolate under the same genus are P. shioyasakiensis and P. gelatinilytica (indicated by the solid line box)

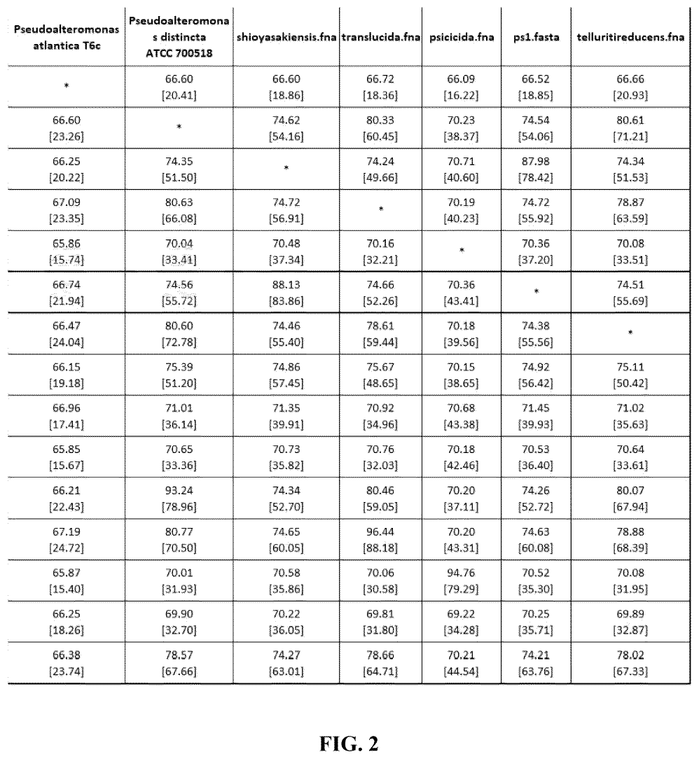

Then, the genomic sequence of the Pseudoalteromonas isolate (ATCC Patent Deposit No. PTA-127701) was compared with 14 other known Pseudoalteromonas species with complete genomic sequences to estimate average nucleotide similarities among them. The results are shown in FIG. 2, in which Pseudoalteromonas shioyasakiensis shares the highest nucleotide similarity (up to around 90%) to the Pseudoalteromonas isolate (ATCC Patent Deposit No. PTA-127701). Typically, two different bacterial isolates collected from two different sources are considered as the same species when their average nucleotide similarities of their genomes are higher than 95%. Thus, the Pseudoalteromonas isolate (ATCC Patent Deposit No. PTA-127701) was considered to be a novel species since there were around 10% differences between the genomic sequences of the isolate and its closest relative P. shioyasakiensis.

### Example 2—Co-Cultivation System of Dunaliella Spp. And Pseudoalteromonas Isolate (ATCC Patent Deposit No. PTA-127701)

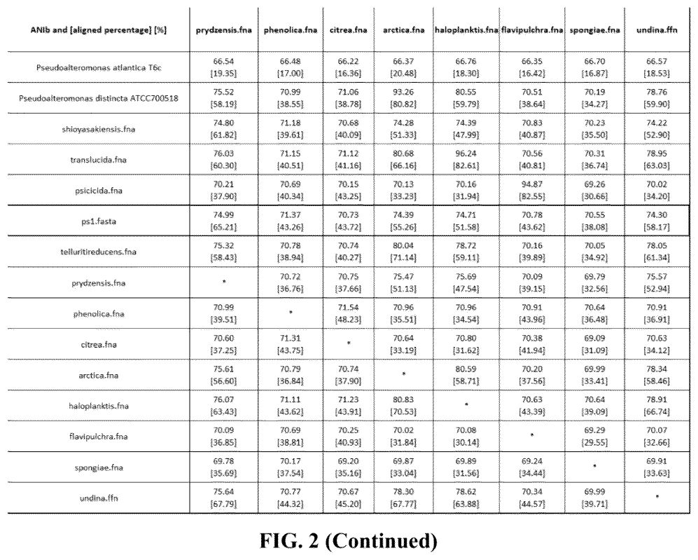

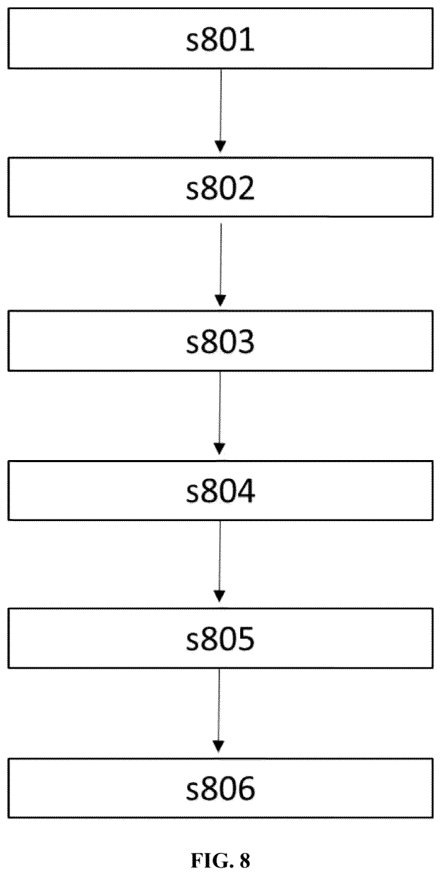

As depicted in FIG. 8, the Pseudoalteromonas isolate (ATCC Patent Deposit No. PTA-127701) was first grown in LB medium prepared in synthetic seawater (29.9 g/L Instant Ocean seasalt, France) overnight to get a bacterial population density equivalent to OD600 around 1 (s801). The isolate was then collected by centrifugation and then washed with the synthetic seawater for 3 times to remove trace amount of bacterial culture medium (s802). Subsequently, the isolate was added into one of the compartments, e.g., in the semi-permeable vessel, which is only permeable to substances with a molecular weight of less than 10 k Da (s803). The semi-permeable vessel added with the isolate was inoculated with D. tertiolecta CCMP364 in either silicate-free K medium or L1 medium (detailed recipes of K and L1 medium are documented in NCMA web site) in a separate compartment (s804). In certain embodiments, an optimal microalgae-to-bacteria ratio of the present disclosure is 1:1000, derived from the most number of cells per mL of the cell culture as shown in FIG. 3A. It was defined as co-culturing 100 ml of the novel isolate (OD600 around 1) with 1 L of D. tertiolecta CCMP364 (10 k microalgae per ml of K medium). In this setup, the cell density of microalgae to bacteria was 1:1000. As seen from FIG. 3B, the microalgal density reached maximum when 1 unit of the novel isolate (ATCC Patent Deposit No. PTA-127701) was inoculated with D. tertiolecta CCMP364, whereas increasing dose over 1 unit did not further promote microalgal growth.

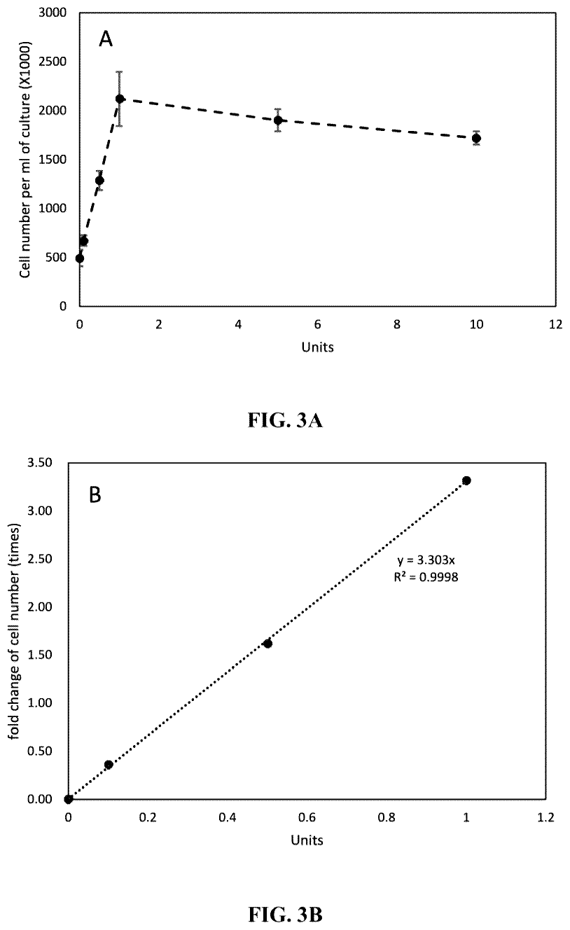

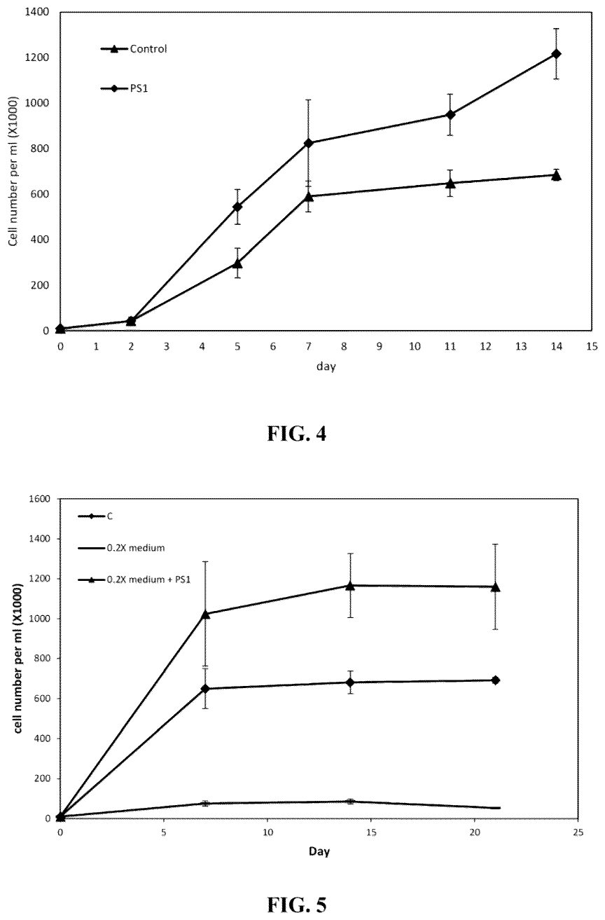

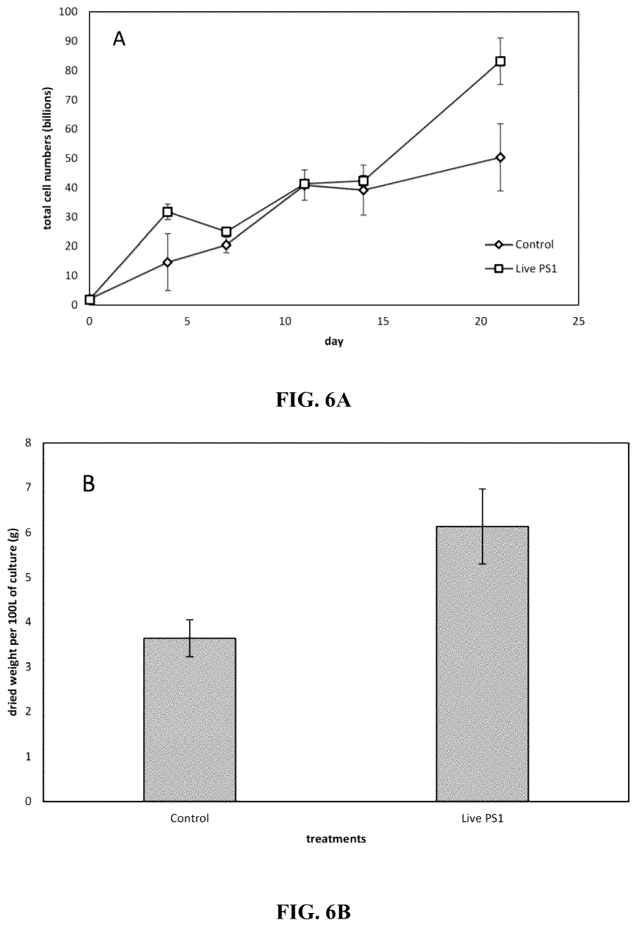

The yields of D. tertiolecta CCMP364 surprisingly increased with a range from around 40% to 3 folds in terms of microalgal densities, and around 20% to 5 folds in terms of dried microalgal biomass weight after 14 days post-inoculation (FIG. 4 and Table 1). The increases of the microalgal densities and dried microalgal biomass depended on the composition of the selected medium (Table 1). The growth enhancing effect of the present isolate (ATCC Patent Deposit No. PTA-127701) on the microalgae in the co-cultivation system was not just observed when using normal K medium, it was also observed when co-culturing D. tertiolecta CCMP364 with the isolate using 5-fold diluted K medium (FIG. 5). The scale of the co-cultivation between D. tertiolecta CCMP364 and the present isolate were further up-scaled from 1 L to 90 L. The microalgal density and dried biomass significantly increased after 21 days (FIGS. 6A-B). After the co-cultivation, the present isolate (ATCC Patent Deposit No. PTA-127701) could be easily separated from the microalgae by removing the semi-permeable vessel (s805) before harvesting the microalgal biomass (s806).

TABLE 1
The effects of the present Pseudoalteromonas isolate
(ATCC Patent Deposit No. PTA-127701) on the growth of three
Dunaliella species: CCMP364, CCMP1320 and STK2:

| | Live bacterium (medium added) | | | | | |
|---|---|---|---|---|---|---|
| | CCMP364 | CCMP364 | CCMP1320 | CCMP1320 | STK2 | STK2 |
| | (K | (L1 | (K | (L1 | (K | (L1 |
| | medium) | medium) | medium) | medium) | medium) | medium) |
| Cell | +327% | +42.8% | +298% | +56.3% | +1252% | +36.3% |
| number | | | | | | |
| (day 14) | | | | | | |
| Biomass | +542% | +22.1% | +138% | +62% | +398% | +40.2% |
| (dry | | | | | | |
| weight) | | | | | | |
| Protein | +520% | +11.9% | +266% | +306% | +856% | +14.3% |
| (yield) | | | | | | |
| CHO | +1095% | −8.4% | +8.8% | +96.6% | +89% | +3.9% |
| (yield) | | | | | | |
| Lipid | +431% | −3.8% | +51.3% | +57.8% | +270% | +56.3% |
| (yield) | | | | | | |

The growth promoting effect of co-culturing the present isolate (ATCC Patent Deposit No. PTA-127701) was further investigated using two other Dunaliella strains, CCMP1320 and STK2. It was found that the co-cultivation of the present isolate (ATCC Patent Deposit No. PTA-127701) could increase the microalgal densities and dried biomass of both Dunaliella spp (Table 1).

### Example 3—Co-Cultivation of Dunaliella spp. with Other Pseudoalteromonas spp.

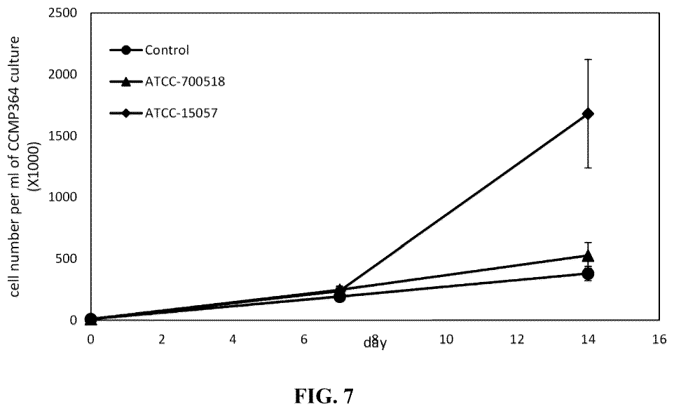

Following the same co-cultivation setup and conditions depicted in Example 2, the effects of other commercially available Pseudoalteromonas species on the growth of Dunaliella spp. have been investigated. It was found that Pseudoalteromonas piscicda (strain ATCC 15057) could promote the growth of D. tertiolecta CCMP364, but Pseudoalteromonas distincta (strain ATCC 700518) could not (FIG. 7).

Although the invention has been described in terms of certain embodiments, other embodiments apparent to those of ordinary skill in the art are also within the scope of this invention. Accordingly, the scope of the invention is intended to be defined only by the claims which follow.

What is claimed is:

1. A method for co-culturing a microalgal species under a genus of Dunaliella with a bacterial strain under a genus of Pseudoalteromonas in a confined co-cultivation environment to enhance growth of the microalgal species comprising:
providing the microalgal species and the bacterial strain to the confined co-cultivation environment; and
providing a culture medium to the confined co-cultivation environment and a set of co-cultivation conditions.

2. The method of claim 1, wherein the microalgal species comprises Dunaliella tertiolecta with an origin of CCMP364 or CCMP1320, or STK2 (Dunaliella sp.) isolated from Sha Tau Kok, Hong Kong in 2013.

3. The method of claim 1, wherein the bacterial strain comprises an isolate under the genus of Pseudoalteromonas having a genome sequence composed of two circular DNAs represented by SEQ ID NOs: 1 and 2, respectively, or any species under the genus of Pseudoalteromonas having a genome sequence with a nucleotide similarity of at least 90% to SEQ ID NOs: 1 and 2.

4. The method of claim 3, wherein the species having the genome sequence with the nucleotide similarity of at least 90% to the genome sequence of the isolate comprises Pseudoalteromonas shioyasakiensis, Pseudoalteromonas gelatinilytica, or Pseudoalteromonas piscicda.

5. The method of claim 1, wherein the bacterial strain comprises the Pseudoalteromonas having been deposited with the American Type Culture Collection under Patent Deposit Designation Number PTA-127701.

6. The method of claim 1, wherein the confined co-cultivation environment comprises at least two compartments each accommodating the microalgal species or the bacterial strain, and the compartment accommodating the bacterial strain is configured to be permeable to substances of less than 10 kDa in molecular weight between any two of the compartments accommodating the microalgal species and the bacterial strain, respectively.

7. The method of claim 1, wherein the microalgal species and the bacterial strain are provided in a cell density ratio of 1:1000.

8. An algal growth enhancing composition comprising a biological agent or an extract thereof, the biological agent comprising a genome sequence composed of two circular DNAs represented by SEQ ID NOs: 1 and 2, respectively, or any genomic sequence having a nucleotide similarity of at least 90% to the genome sequence represented by SEQ ID NOs: 1 and 2.

9. The composition of claim 8, wherein the biological agent is a bacterial strain under a genus of Pseudoalteromonas.

10. The composition of claim 8, wherein the bacterial strain comprises Pseudoalteromonas shioyasakiensis, Pseudoalteromonas gelatinilytica, Pseudoalteromonas piscicda, or any Pseudoalteromonas spp. comprising a genomic sequence having a nucleotide similarity of at least 90% to the genome sequence represented by SEQ ID NOs: 1 and 2.

11. The composition of claim 10, wherein the bacterial strain comprises Pseudoalteromonas having been deposited with the American Type Culture Collection under Patent Deposit Designation Number PTA-127701.

12. The composition of claim 8, wherein the algal growth is a growth of microalgal species under a genus of Dunaliella.

13. The composition of claim 12, wherein the microalgal species comprises Dunaliella tertiolecta with an origin of CCMP364 or CCMP1320, or STK2 (Dunaliella sp.) isolated from Sha Tau Kok, Hong Kong in 2013

14. The composition of claim 8, wherein the extract of the biological agent comprises a culture medium, cellular and molecular contents of the biological agent.

15. The composition of claim 14, wherein the extract has a molecular weight of less than 10 kDa.

16. A method for increasing biomass production yield from a microalgal species comprising:
co-culturing the microalgal species with a bacterial strain under a genus of Pseudoalteromonas, wherein the bacterial strain comprises a genome sequence composed of two circular DNAs represented by SEQ ID NOs: 1 and 2, respectively, a genome sequence having a nucleotide similarity of at least 90% to the genome sequence represented by SEQ ID NOs. 1 and 2, or the bacterial strain comprises the Pseudoalteromonas having been deposited with the American Type Culture Collection under Patent Deposit Designation Number PTA-127701; or
contacting the microalgal species with an extract of the bacterial strain, wherein the extract comprises a culture medium, cellular and molecular contents of the bacterial strain.

17. The method of claim 16, wherein the microalgal species comprises Dunaliella tertiolecta with an origin of CCMP364 or CCMP1320, or STK2 (Dunaliella sp.) isolated from Sha Tau Kok, Hong Kong in 2013.

18. The method of claim 16, wherein the bacterial strain comprises Pseudoalteromonas shioyasakiensis, Pseudoalteromonas gelatinilytica, Pseudoalteromonas piscicda, or any Pseudoalteromonas spp. comprising a genomic sequence having a nucleotide similarity of at least 90% to the genome sequence represented by SEQ ID NOs: 1 and 2.

19. The method of claim 16, wherein the bacterial strain comprises Pseudoalteromonas having been deposited with the American Type Culture Collection under Patent Deposit Designation Number PTA-127701.

20. The method of claim 16, wherein said co-culturing is performed in a confined co-cultivation environment.

21. The method of claim 16, wherein the confined co-cultivation environment comprises at least two compartments each accommodating the microalgal species or the bacterial strain, wherein the compartment accommodating the bacterial strain is configured to be permeable to substances of less than 10 kDa in molecular weight between the two compartments accommodating the microalgal species and the bacterial strain, respectively.

22. The method of claim 16, wherein the extract has a molecular weight of less than 10 kDa.

23. A co-culture system comprising a microalgal species under the genus of Dunaliella and an isolate of bacterial strain under the genus of Pseudoalteromonas, the isolate of bacterial strain under the genus of Pseudoalteromonas comprising a genome sequence composed of two circular DNAs represented by SEQ ID NOs: 1 and 2; or the bacterial strain comprises the Pseudoalteromonas having been deposited with the American Type Culture Collection under Patent Deposit Designation Number PTA-127701.
